(19) United States
(12) Patent Application Publication
(10) Pub. No.: US 2019/0211343 A1
(43) Pub. Date: Jul. 11, 2019

(54) PROCESS FOR THE TRANSFORMATION OF BASIDIOMYCETES BY INCLUSION OF GENETIC MATERIAL IN PROTOPLAST OBTAINED FROM MYCELIUM, USING POLYETHYLENE GLYCOL AS AN ADJUVANT

(71) Applicant: POLYBIO, SOCIEDAD ANÓNIMA PROMOTORA DE INVERSIÓN DE CAPITAL VARIABLE, GUANAJUATO (MX)

(72) Inventors: AXEL ALEJANDRO GOMEZ-ORTIGOZA AGUIRRE, GUANAJUATO (MX); ALEXIS MANUEL GOMEZ-ORTIGOZA AGUIRRE, GUANAJUATO (MX); BARBARA GONZALEZ ROLON, GUANAJUATO (MX); FRANCISCO FEDERICO NUNEZ DE CACERES GONZALEZ, GUANAJUATO (MX)

(73) Assignee: POLYBIO, SOCIEDAD ANÓNIMA PROMOTORA DE INVERSIÓN D E CAPITAL VARIABLE, GUANAJUATO (MX)

(21) Appl. No.: 16/125,998

(22) Filed: Sep. 10, 2018

(30) Foreign Application Priority Data
Jan. 9, 2018 (MX) MX/A/2018/000319

(51) Int. Cl. C12N 15/80; C12N 9/26; C12R 1/645

(52) U.S. Cl. CPC C12N 15/80; C12Y 302/01; C12R 1/645; C12N 9/2411

(57) ABSTRACT

The presented invention refers to a process to genetically modify fungi of basidiomycetes in 3 steps: a) collection of protoplasts, that includes the incubation of mycelia with enzymes of digestion in combination with the use of an osmotic agent to guard and increase rates of recovery; b) transformation (modified genetics), that includes incubation of the genetic material with polyethylene glycol in combination with an osmotic agent as a guard and to increase the rate of recovery, and c) recovery and selection of the transformants. Said process has more efficiency of transformation to the process currently used, mainly because it has a higher rate of recovery and faster processing time.

### TECHNICAL FIELD OF THE INVENTION

The present invention refers to the process for genetically modifying protoplasts of basidiomycetes mushroom, and specifically concerns one of these processes based in digestion enzymes, using polyethylene glycol as adjuvant for the introduction of genetic material and an osmotic agent as a protector, delivering a high efficient transformation process with high versatility of application to the vast majority of different species of basidiomycetes.

### Antecedents of the Invention

In this document the terms that follow have their definitions as indicated.

Basidiomycetes refers to the set of species from the phylum basidiomycota inside of the Fungi Kingdom, which produces basidiums with basidiospores.

Species should be understood as the basic unit of the biological classification (taxonomy).

Protoplast should be defined as a plant cell, bacteria or fungus that has lost total or part of the cell wall.

Plasmid should be defined as a molecule of DNA (deoxyribonucleic acid) extra-chromosomal that is generally circular in shape that replicates and transmits independent of the chromosomal DNA.

Adjuvant should be defined as the element and/or system that facilitates the inclusion of genetic material to the cell.

In general terms, the transformation process (genetic modification) of any type of cell depends on an adjuvant that transports the genetic material to the interior of the same. So physical, chemical or biological processes are used to cross the cell wall (if is the case), as well as the cytoplasmic membrane. By the nature of said processes, said cell suffers damage that is in many cases irreparable, causing the cellular death (lysis) and with it preventing the transformation process on this cell. Since thousands or millions of cells are subject to this process, a percentage of them survive the process, making the desired genetic modifications. One way to express the efficiency of said process is by the transformation efficiency, which quantifies how many cells submitted to the process are capable to survive and incorporate the genetic modification in their genome. Therefore, the way that a process harms a particular cell is inversely proportional to the efficiency of the transformation.

The reason that the group of the basidiomycetes is of special interest, environmental, economic, social, technological, and scientific, falls into their application as: food, gourmet food, alternative medicine element, bioremediation agent, source of industrial interest molecules, as biomaterial, among other applications.

Up to this time, there are various processes for achieving the genetic modification of fungi, among the more widely known are the tissue incubation such as spores, protoplasts, and mycelium with Agrobacterium tumefaciens, as can be found on “Agrobacterium-Mediated Transformation of Fusarium oxysporum: An Efficient Tool for Insertional Mutagenesis and Gene Transfer”, (Mullins et al., Phytopathology 2001. February 91 (2): 173-80), which describes the genetic modification of a deuteromycetes using Agrobacterium tumefaciens, but does not describe the transformation of basidiomycetes; “Efficient GFP expression in the mushrooms Agaricus bisporus and Coprinus cinereus requires introns”, (Burns et al., Fungal Genet Biol, 2005. 42: 191-199), which describes the genetic modification of two basidiomycetes using Agrobacterium tumefaciens but does not describe the efficiency of the process; “Agrobacterium-mediated transformation of the winter mushroom Flammulina velutipes”, (Cho et, Mycobiology, 2006. 34:104-107), which describes the genetic modification of a basidiomycete using Agrobacterium tumefaciens, but is unspecific as to the incision site and the number of copies inserted, in addition to not reporting the efficiency of the transformation, by reporting only the transformation frequency, without cellular quantification; “Agrobacterium-mediated” delivery of marker genes to Phanerochaete chrysosporium mycelial pellets: “A model transformation system for white-rot fungi” (Sharma et al., Biotechnol Appl Biochem, 2006. 43:181-186), which describes the genetic modification of a basidiomycete using Agrobacterium tumefaciens and required the use of acetosyringone as additives for achieving a transformation, which increases the complexity grade of the process, in addition that it does not report the transformation efficiency, only reports the transformation frequency, without the cell quantification; “An efficient tool for random insertional mutagenesis: Agrobacterium tumefaciens-mediated transformation of the filamentous fungus Aspergillus terreus” (Wang et al., Journal of Microbiological Methods, 2014 98:114-118), which describes the genetic modification of an Ascomycete using Agrobacterium tumefaciens, but requires the use of acetosyringone to achieve the transformation increasing the complexity grade and expense of the process; “Agrobacterium tumefaciens-mediated transformation in the entomopathogenic fungus Lecanicillium lecanii and development of benzimidazole fungicide resistant strains”; (Zhang et al., J)

Microbiol Meth, 2014. 105:168-173), which describes the genetic modification of an ascomycete using Agrobacterium tumefaciens and requires the use of acetosyringone as an additive to achieve the transformation which increases the degree of complexity and makes the process more expensive.

In general, the use of Agrobacterium tumefaciens as adjuvant in the Introduction of genetic material presents different disadvantages like the unspecified insertion site and the lack of control on the number of copies inserted, caused by the pathogenic nature of the aforementioned adjuvant. If it can reach important efficiency transformations, it will require liable additives and be expensive, which adds complexity to the process, increasing the unspecified site of incision and the lack of control on the number of copies inserted, that can generate an undesired muted effect of genes or deleterious modifications in genes crucial for the survival of the micro-organism. In addition, it is also not applicable to a wide range of fungal species and has the issue of the elimination of the bacteria once the genetic material has been transferred, which can result in tedious work and will require a lot of time. In other words, a disadvantage to this process is that it generates secondary residues that are difficult to eliminate, such as the Agrobacterium tumefaciens cells remaining after the process. And does not solve the problem of low efficiency in an integral manner and does not represent an efficient process.

Another process used is the tissue bombardment, mainly spores, is by bioballistic as can be seen in “Stable genetic transformation of the ectomycorrhizal fungus Pisolithus tinctorius’” (Rodriguez—Tovar et al., J Microbiol Methods, 2005. 63:45-54), which describes the genetic modification of a basidiomycete by bioballistics and indicated obtaining a lower transformation efficiency with this process that uses Agrobacterium tumefaciens; “Transient transformation of the oblgate biotrophic rust fungus Uromyces fabae using biolistics” (Djulic et al., Fungal Biology, 2011. 115:633-642), which describes the genetic modification of a basidiomycete by bioballistics and indicates only obtaining a transient transformation, since it was not able to integrate the modification to the genome; “Biolistic Transformation for Delivering DNA into the Mitochondria” (Montanari et al., Genetic Transformation Systems in Fungi, 2015. 1:101-117), which describes the genetic modification of the mitochondria via bioballistics in fungus in general and indicates an extremely inefficient process that destroys 99% of the cells subject to the process and in the 1% that survive not all of them incorporate the modification; “Transformation of Zygomycete Mortierella alpina Using Biolistic Particle Bombardment” (Sakuradani et al., Genetic Transformation Systems in Fungi, 2015. 1:135-140), which describes the genetic modification of a zygomycete by bioballistics, but does not report the level of efficiency of the transformation using the aforementioned process.

In general, the use of bioballistics as an adjuvant to the introduction of genetic material presents various disadvantages, such as the low transformation efficiency, due to the mechanical damage by the impact generated on the cell wall, in addition transferred genetic material can be subject to damage by impact in the same way. Additionally, there is another disadvantage that depends on the availability of the specialized equipment and liable supplies of high purity that are related to the process, making it a difficult to handle process.

In addition, the amount of time it takes to execute the process is slow, since the targeted cells must be prepared prior to this process.

It can be found in the art, a process that uses electric pulses to induce pores in the cell wall, known as Electroporation, which can be found in “Transformation of filamentous fungi with plasmid DNA by Electroporation” (Richey et al., Phytopathologand, 1989 79:844-847), which describes the genetic modification of two Ascomycetes by Electroporation, but reports very low efficiency; “An improved protocol for the preparation of yeast cells for transformation band electroporation” (Thompson et al., Yeast, 1998. 14:565-71) that describes the genetic modification of two ascomycetes by Electroporation, but requires lithium acetate and dithiothreitol as additives to achieve the transformation, which increases the grade of complexity and amount of expenses of the process; “Electroporation Mediated DNA Transformation of Filamentous Fungi” (Chakrabaty, Genetic Transformation Systems in Fungi, 2015. 1:67-79), which describes the genetic modification of an ascomycetes for Electroporation, but reports low efficiency.

In the case of the process based in Electroporation, the efficiency of the transformation is decreased because the type of cell that attempts to transform (fungal) and the electrophysiological properties interfere with the cell recovery process after the introduction of pores on the cell wall, which at the same time decreases the transformation efficiency. In addition, the execution time of the process is delayed because the targeted cells should already be prepared to be competent to use in said process.

Another process used is the sonoporation that increases the permeability of the cell membrane allowing the introduction of genetic material to the cell as can be confirmed in “A novel and highly efficient method for genetic transformation of fungi employing shock waves” (Magana-Ortiz et al., Fungal Genet Biol, 2013. 56:9-16), which describes the genetic modification of two ascomycetes, a deuteromycete, and a basidiomycete, that even if it obtains interesting transformation efficiently, requires an experimental acoustic cavilacion equipment that is not a line equipment that can be found on the market, and the difficulty to access said adjuvant and thus the process remains as such. This is to say that the process of Sonoporacion also limits by way of the structural modification of the cell wall due to acoustics cavitation. Said process is based in physical properties of the cell and requires equipment that is specialized to generate an ultrasound wave while keeping the integrity of the target cell; the process is expensive, complex and difficult to reproduce. In addition, the time of execution of the process is delayed because the targeted cell must be prepared to be competent for this process.

TABLE 1 presents a comparison of the efficiencies of reported transformations on the state of the art. It is pointed out that some of the efficiencies which are expressed like a percentage of transformed tissue with respect to the quantity of tissues subject to the process, represents a transformation frequency and not a transformation efficiency, since the authors do not report the tissue size or the feasibility of the same (cell quantification), because in these cases the number of cells present is very high and the number of cells transformed is very low, resulting in low efficiencies of transformation. The previous generates ambiguity in respect to the time of comparing said process of transformation with processes where efficiencies of transformation are described as very high, expressed as the amount of cells transformed in respect to the total amount of cells submitted to the process, and this must be taken into consideration.

TABLE 1
Efficiencies of transformations reported in cited literature.

| | Transformation | | |
|---|---|---|---|
| Source | efficiency | Adyuvant | Phylum |
| Mullins et al | 500 of every 1,000,000 of | Agrobacterium | Deuteromycetes |
| | cells are transformed | tumefaciens | |
| Cho et al. | 16% of exposed tissues | Agrobacterium | Basidiomycetes |
| | are transformed | tumefaciens | |
| Sharma et al. | 36-48% exposed tissues | Agrobacterium | Basidiomycetes |
| | are transformed | tumefaciens | |
| Wang et al. | 350 of every 1,000,000 of the | Agrobacterium | Ascomycete |
| | cells are transformed | tumefaciens | |
| Zhang et al. | 25 of every 1,000,000 of the | Agrobacterium | Ascomycete |
| | cells are transformed | tumefaciens | |
| Rodríguez- | 10% exposed tissues | Bioballistics | Basidiomycetes |
| Tovar et al. | are transformed | | |
| Djulic et al. | 45 of every 1,000,000 of the | Bioballistics | Basidiomycetes |
| | cells are transformed | | |
| Montanari et | 10 of every 1,000,000 of the | Bioballistics | — |
| al. | cells are transformed | | |
| Richey et al. | 1 of every 1,000,000 of the | Electroporation | Ascomycete |
| | cells are transformed | | |
| Thompson et | 104 of every 1,000,000 of the | Electroporation | Ascomycete |
| al. | cells are transformed | | |
| Chakraborty. | 6 of every 1,000,000 of the | Electroporation | Ascomycete |
| | cells are transformed | | |
| Magaña-Ortiz | 5,333 of every 1,000,000 of the | Sonoporation | Basidiomycetes |
| et al. | cells are transformed | | |
| Magaña-Ortiz | 6,050 of every 1,000,000 of the | Sonoporation | Ascomycete |
| et al. | cells are transformed | | |
| Magaña-Ortiz | 12,000 of every 1,000,000 of | Sonoporation | Deuteromycete |
| et al. | the | | |
| | cells are | | |
| | transformed | | |

It is also important to mention that there are transformation processes for various phylum, that should only be considered for comparison purposes with the present invention, in cases in which the basidiomycete transformations are present in order to generate a coherent comparison within the literature and the submitted process of that particular invention.

### Objectives of the Invention

In view of the difficulties and limits of the known conventional processes, it is an objective of this invention to supply a new process for genetically modifying fungi protoplasts from the basidiomycetes group.

Another objective of this invention is to provide a process to genetically modify protoplasts of basidiomycetes that does not involve virulent elements for the introduction of genetic material, thus increasing the efficiency of transformations without increasing the unspecific in the insertion site nor the lack of control in the number of copies.

As presented, this invention has the objective to provide a process to genetically modify protoplasts of basidiomycetes that does not include mechanical elements in the introduction of the genetic material, which increases the transformation efficiency with respect to the bioballistic process without increases to the rate of damage to the genetic material involved nor the unspecific.

Another objective of the presented invention is to provide a process to genetically modify basidiomycetes protoplasts which makes use of a biochemical adjuvant and not physical ones, preventing direct damage to the cell and increasing the efficiency of the transformation without decreasing the recovery rate of the subject cell.

Another objective of the presented invention is to provide a process to genetically modify the basidiomycetes protoplasts that is based in using digestive enzymes to obtain the above-mentioned protoplasts.

Another objective of the presented invention is to provide a process of genetically modifying basidiomycetes by a process: very replicable and short time span to complete, capable of being completed with the equipment and supplies of a basic laboratory, without the need to use specialized equipment, custom made, difficult to use, of low availability.

It is another objective of the presented invention to provide and process to genetically modify protoplasts of basidiomycetes which permits a high efficiency transformation.

These and other objectives will be evidently clear with the following descriptions and accompanying figures, without being limited but illustrative of the process of the presented invention.

### BRIEF DESCRIPTION OF THE FIGURES

FIG. 1 is a flow diagram of the complete transformation process of basidiomycetes by insertion of the genetic material in protoplasts obtained via the mycelium.

FIG. 2 shows ganoderma applanatum protoplasts before and after the transformation process.

FIG. 2(a) shows ganoderma applanatum protoplasts before the transformation process.

FIG. 2(b) shows ganoderma applanatum protoplasts after the transformation process.

FIG. 3 shows trametes elegans protoplasts before and after the transformation process.

FIG. 3(a) shows trametes elegans protoplasts before the transformation process.

FIG. 3(b) shows trametes legans protoplasts after the transformation process.

FIG. 4 shows ganoderma lucidum protoplasts before and after the transformation process.

FIG. 4(a) shows ganoderma lucidum protoplasts before the transformation process.

FIG. 4(b) shows ganoderma lucidum protoplasts after the transformation process.

FIG. 5 is a flow diagram showing the steps of collection of protoplasts via mycelium.

FIG. 6 is a flow diagram of the step of processing the genetics of the protoplasts.

FIG. 7 is a flow diagram of the step of recovery, selection and proliferation of the transformed cells.

### BRIEF DESCRIPTION OF THE INVENTION

The present invention refers to a process for the genetic modification of basidiomycetes fungi that includes 3 steps, starting with obtaining protoplasts, followed by the transformation (genetic modification), ending with the recovery, selection, and proliferation of the transformants. The step of collecting the protoplasts includes: short incubation periods of the mycelia with digestions enzymes (ex: Cellulase and Pectinase) in combination with the use of an osmotic agent (ex. Sorbitol) as a protector for increasing the recovery rate. The transformation step includes: a brief incubation of the genetic material (ex. bacterial plasmids) with use of polyethylene glycol in combination with an osmotic agent (ex. Sorbitol) as a protector for increasing the recovery rate. Said process includes a bigger transformation efficiency than the process described by the state of the art, mainly because it has a recovery rate that is higher and faster, reducing the process time. Additionally, there is a versatility in the number of species of basidiomycetes that can be transformed by the process.

### DETAILED DESCRIPTION OF THE INVENTION

The presented invention describes a process that is highly efficient in the introduction of genetic material to fungal cells from the phylum basidiomycetes. The previous statement is achieved by substituting the mechanical, physical and biological convention process of the state of the art, with a biochemical process that utilizes digestion enzymes that are able to degrade the cell wall in a controlled and gradual manner, allowing the designated cell to be introduced with genetic material with the least damage possible, incrementing to recuperation rate and therefore the transformation efficiency. Once the designated cell loses total or part of its cell wall and converts into a protoplast, the use of polyethyleneglycol as adjuvant helps the entrance of genetic material, since its biochemical properties permit the dissolution of the genetic material through the cytoplasmic membrane of the cell without damaging it, increasing the recovery rate of the cells that have gone through the process and also the transformation efficiency via the process. Simultaneously, both of the step of generation of the protoplasts as well as the step of the introducing of genetic material, use an osmotic agent to decrease the osmotic pressure exerted over the cytoplasmatic membrane, protecting the objective cells and increasing the recovery rate and therefore the efficiency of the process. As noted, the elements that allow the presented invention to increase the transformation efficiency are: the increase of the recovery rate via protection of the cell, as well as the use of a biochemical adjuvant (polyethylene glycol) does not do damage to the objective cell during the process of introducing genetic material.

Highlighted that the nature of the process allows it to be successfully applied to a broad number of basidiomycetes species because it is not depending on mechanical, physical, of strictly biological factors particular of each species, since it uses a biochemical adjuvant accompanied by an osmotic pressure regulator in conjunction with the use of bare cells (protoplasts). Said elements are easily applicable to a series of basidiomycetes species. As can be seen on Table 2, the presented process increases transformation of three different basidiomycetes species with high efficiency.

TABLE 2
Efficiencies of transformation of the presented invention

| | Transformation | | |
|---|---|---|---|
| Species | efficacy | Adyuvant | Phylum |
| Ganoderma | 81,000 of every 1,000,000 | Polyethylene | Basidiomycetes |
| applanatum | cells are transformed | glycol | |
| Trametes | 109,000 of every | Polyethylene | Basidiomycetes |
| elegans | 1,000,000 | glycol | |
| | cells are transformed | | |
| Ganoderma | 194,000 of every | Polyethylene | Basidiomycetes |
| lucidum | 1,000,000 | glycol | |
| | cells are transformed | | |
| Average | 128,000 of every | Polyethylene | Basidiomycetes |
| | 1,000,000 | glycol | |
| | cells are transformed | | |

Comparing table 1 and 2 can be noted that there is a difference in the transformation efficiency between the procedures of the prior art and the present invention, being higher the ones of the present invention. In addition, can be corroborated doing the same comparison, that the presented invention achieves the basidiomycetes transformation in a versatile manner in three different species in comparing with the state of the art that does not achieve to transform more than two species of basidiomycetes by the same process.

The methodology used for the introduction of genetic material (ex. bacterial plasmids) to fungi protoplasts, using polyethylene glycol as an adjuvant and an osmotic agent (ex. Sorbitol) as a protector of the process of invention, is explained below.

Along the following explanation are mentioned compositions and concentrations as presented in Tables 3, 4, 5 and 6, that express an optimum range of operation for the process but are not limited on how many there are of the concentrations of the components. In order words, they serve to illustrate the preferred concentration range of each component, as well as examples of the components preferred use for the process, but do not denote a limit in the specified components, or the specific concentrations of each component exemplified for the execution process. In other words, the objective of the present invention is the process as is and not the used tools, concentrations, or examples of the elements and components involved.

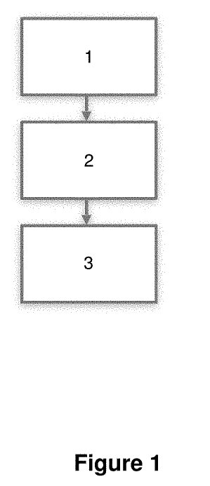

The process of this invention is shown schematically in FIG. 1 and includes the following steps:

(1) obtaining the protoplasts (1) using digestion enzymes, originated from the mycelium grown in petri dishes with a medium semi-solid and an osmotic agent as protector,

(2) genetic transformation of the protoplasts (2) by the incubation with genetic material and polyethylene glycol as an adjuvant to inserting of said genetic material on the protoplasts obtained during the previous phase, as well as an osmotic agent as protector,

(3) recovery and regeneration (3) in liquid substrate of the transformed cells and posterior seeding and proliferating in a semi-solid medium for the selection of those protoplasts which have been transformed and are expressing the transgenes of interest.

All the operations must be completed in sterile conditions to avoid pollution with microorganisms and/or nucleic acids that interfere with the process.

To continue, a description of each one of the steps of the process of invention:

### Obtaining the Protoplasts

The purpose of the step of obtaining the protoplasts (1) in FIG. 1 is to get protoplasts that have high osmotic resistance to the posterior biochemical manipulation that will be given during the process. The protoplasts are cells that do not have cell walls, facilitating the entrance of genetic material but at the same time lack this protection and stability, thus, they are very delicate and tend to be consequential to experimenting cellular lysis. These characteristics also make it difficult and slow their recovery and subsequent proliferation. Thus, the use of an osmotic agent (ex. Sorbitol) favors the stability of these fragile cells, and therefore increases the recovery rate and transformation efficiency.

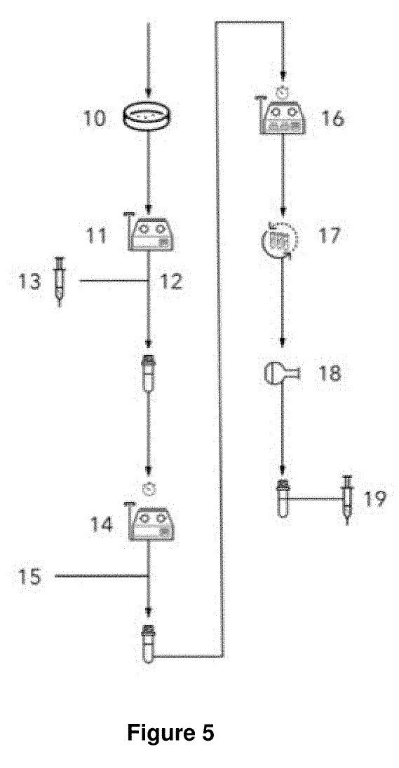

The steps following at this stage are illustrated on FIG. 5 and are:
I. grow (10) mycelium of the desired species to be modified, in petri dishes with semi-solid medium, with the composition w/v (w/v) shown in Table 3, using demineralized water as a solvent and sterilizing in an autoclave the solution:

TABLE 3
Composition of the medium culture for the
proliferation of the mycelium

| Component | Concentration (w/v) |
|---|---|
| Carbon source | 5-35% |
| Saccharide | 0.1-15% |
| Gelling agent | 0.5-10% |

The composition of the culture medium for the proliferation of mycelia presented in Table 3 should incorporate a carbon source, where this one is a one with high polysaccharides content, that is selected from the group including potato and malt extracts, a monosaccharide, where it is of fast metabolism, selected from the group that includes dextrose, maltose, and dextrin and a gelling agent, selected from the group including fitagel, alignate, and agar, all the components previously mentioned should be incorporated in an aqueous solution. The function of the carbon source is to provide carbon to the growing fungi cells. It was opted for a carbon source with high content of polysaccharides given the preferences of the fungi cells for development over substrates with high content of amylase, amylopectin, and maltodextrin such as potato and malt respectively. Additionally, the carbon source is accompanied with a monosaccharide with the objective of favors, through a carbon source of fast metabolism, exponential growth of the fungi cells. The gelling agent, provides a semi-solid consistency and gives support to the mycelium growth.
II. Grow (11) the strains at a temperature of 15-30° C. in dark conditions during the 7 days or until the mycelium completely covers the petri dish.
III. Place (12) the mycelium equivalent to ⅛ of the plate in an Eppendorf tube of 2 mL avoiding carrying the semi-solid culture medium.
IV. Add (13) to the tube a volume, equal to the weight of the mycelium, of an osmotic agent solution 10-30% (w/v) for regulating the osmotic pressure, following the displayed composition in Table 4:

TABLE 4
Composition of the solution of the osmotic agent

| Component | Concentration (w/v) |
|---|---|
| Osmotic agent | 10-30% |

The composition of the osmotic agent solution on Table 4 includes an osmotic agent selected from the group that includes sorbitol and citric acid, both in aqueous solutions. Preferably sorbitol should be used, given its specific osmotic qualities.
V. Submit the tube to a first incubation to obtain the protoplasts (14) at 20-31° C. for 20-40 minutes.
VI. Add (15) 1.5 volume of digestion solution to the tube, with the composition w/v shown in Table 5 and adjust the pH to 5.5-6.3:

TABLE 5
Digestion solution composition

| Component | Concentration |
|---|---|
| Glucan digestion enzyme | 1-10% (w/v) |
| Chitin digestion enzyme | 1-5% (w/v) |
| Osmotic agent | 0.1-1M |
| Shock absorbing agent | 10-100 mM |
| Cytoplasmic membrane stabilizing agent | 1-10 mM |

The digestion solution composition provided in Table 5 should incorporate a glucan digestion enzyme, selected from the group including the cellulase, cellobiohydrolase, and the glucanase, a chitin digestion enzyme, selected from the group that includes the pectinase, the polygalacturonase, and the chitinase, an osmotic agent, selected from the group that includes the sorbitol and citric acid, a shock absorbing agent, selected from the group that includes HEPES, Tris, and TAE, and a cytoplasmic membrane stabilizing agent, which is selected from the group that includes calcium chloride, with all the previous components in an aqueous solution and adjusted to a pH to 5.7-6.1. The function of the glucan digestion enzyme is to degrade the exterior layer of the glucan present in the fungi cell wall. It is preferable to use cellulase or glucanase due to its ability to degrade the glucan with high efficiency. The function of the chitin digestion enzyme is to degrade the internal layer of the chitin present in the fungi cell wall, it is preferable to use Pectinase or chitinase due to its ability to degrade chitin with high efficiency. The previous steps allow for the generation of bare cells or protoplasts that fully or partially lack cell walls, allowing the access to the genetic material. It Includes the osmotic agents, the cytoplasmic membrane stabilizer, and the shock absorber, for protection of the cell from osmotic damage and to cushion the pH of the solution respectively, increasing the survival rate of the objective cells and favoring the good operation of the digestion enzymes.
VII. Submit the tubes to a second incubation for obtaining protoplasts (16) to 25-37° C. with a agitation of 120-170 rpm by a period of 20-45 minutes.
VIII. Centrifuge (17) the mixture at 1000-1800 rpm for 1-5 minutes.
IX. Discard (18) 70-90% of the supernatant.
X. Re-suspend (19) the rest of the volume in 50-100 micro liters of solution of the osmotic agent, submitted in Table 4.

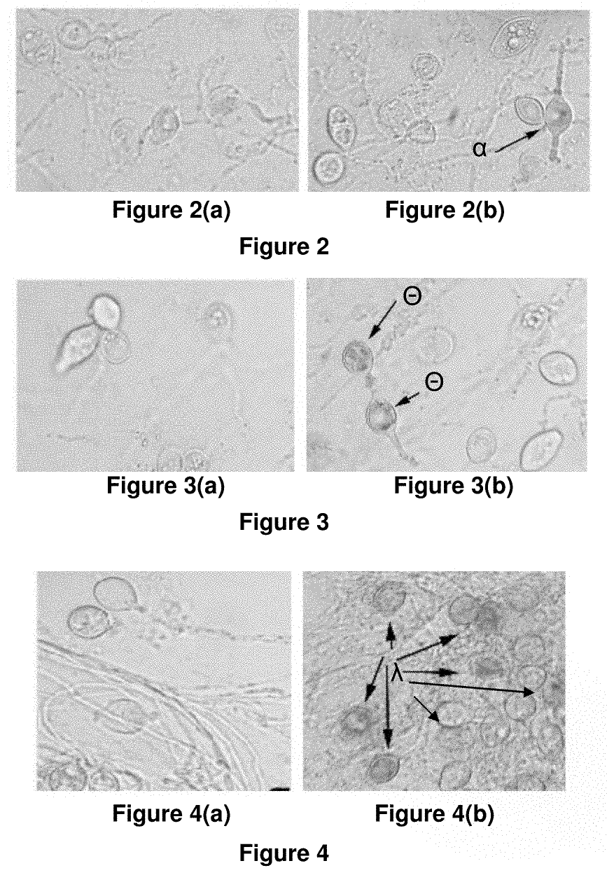

To check the successful and stable collection of protoplasts, takes 10 micro liters of the remaining solution of the last step (19) and observe under the microscope, corroborating the presence of the same, as is seen on the FIG. 2(a) for ganoderma applanatum, on the FIG. 3(a) for trametes elegans, and in FIG. 4(a) to ganoderma lucidum.

### Genetic Transformation of Protoplasts

The objective of this step is the genetic transformation of the protoplasts (2) in FIG. 1, is the genetic modification (transformation) of the resulting protoplasts from the collection step of protoplasts (1) in FIG. 1. The genetic transformation implies a physical transfer of genetic material to the inside of the objective cell, it is important that these protoplasts find the osmotic base conditions ideal, promoting the integrity of the cytoplasmic membrane and the survival of the objective cell.

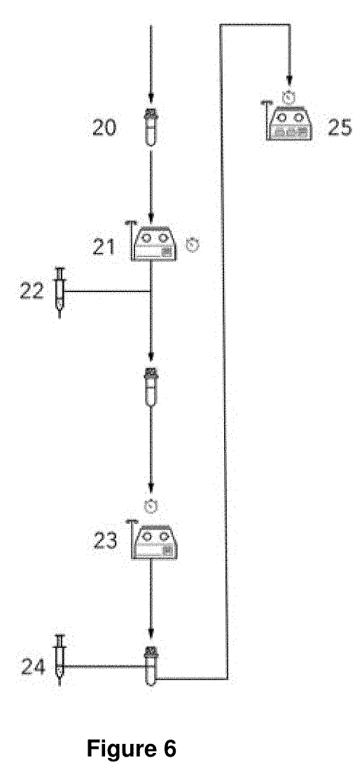

The following steps are illustrated in FIG. 6 and are:

I. add (20) to the tube with protoplasts obtained in the previous step (19), 1-100 micro grams of the genetic material that is to be introduced. The genetic material should include a selection mechanism capable of discerning between the cells that are received and integrated the genetic material in their genomes and the one which does not.

II. Submit the tubes to the first incubation period for the genetic transformation of the protoplasts (21) for 1-10 mins to an environmental temperature (20-25° C.).

III. Add (22) 2-10 micro liters of the adjuvant solution as presented in Table 6:

TABLE 6
Composition of the solution of adjuvant

| Component | Concentration (w/v) |
|---|---|
| Polyethylene glycol | 20-50% |
| Osmotic agent | 10-30% |

The composition of the adjuvant solution submitted in Table 6 should include polyethylene glycol and an osmotic agent, selected from the group that includes the sorbitol and citric acid, both in aqueous solutions. The function of the polyethylene glycol is to act as an adjuvant, increasing the permeability of the genetic material through the cytoplasmic membrane and facilitating the genetic transformation. The osmotic agent acts as a protector from osmotic damage to the cell.

IV. submit the tubes to a second incubation for the genetic transformation of protoplasts (23) to 25-37° C. during 5-15 minutes.

V. Add (24) 1-2 mL of the solution of osmotic agent osmotic as presented in Table 4.

VI. Submit the tubes to a third incubation for the genetic transformation of protoplasts (25) during 40-60 hours in the dark to 15-30° C.

This shows the transformation of basidiomycetes by a selection mechanism presented in the genetic material that should include genetic elements capable of discerning between the cell that received and integrated the genetic material in its genome and the one that did not. Said selection mechanism should preferably be made up of a destructive selection element and a not destructive selection element, allowing for corroboration of the affectivity of this step in a serial manner. Preferably, as the destructive selection element is used, a system based on the gene gusA that provides the objected cell the ability of producing the protein β-glucuronidase, converting the substract X-gluc to a product (chloro-bromoindigo) of blue color and through a histochemical analysis, can be corroborated with the presence of the genetic material on the objective cell. In cases where the protoplasts have been successfully modified and they are expressing the introduced genetic material, including the gene that encodes β-glucoronidase and the genetic material of interest, the protoplasts should show a blue color after the incubation with the substrate X-gluc, as is illustrated in FIG. 2 for Ganoderma applanatum (α), in FIG. 3 for Trametes elegans (Θ) and in FIG. 4 for Ganoderma (λ). The arrows indicate the cells that present said blue color. Given that this procedure is destructive and eliminates the cell subjected to histochemical analysis, it is preferable to add a second selection mechanism that it is not destructive. Preferably, a selection antibiotic, for example hygromycin B, can be incorporated to the gene hph as part of the genetic material to be introduced, same that provides resistance to hygromycin B, allowing the resulting protoplasts to grow, in either a liquid culture medium or a semi-solid medium in presence of hygromycin B and then, to select the cell that receives the integrated genetic material in its genome in place of the ones that cannot do that. In other words, by incorporating the two selection elements previously mentioned (destructive and not destructive) inside the genetic material to be introduced, it is possible to corroborate if the present step of transformation was successful in the introduction of the genetic material of interest in the species of basidiomycete that are sought to be modified.

FIG. 2 shows the Ganoderma applanatum protoplasts being submitted to the step of genetic modification, FIG. 2(a) and at the same time the protoplasts that accepted the genetic material and present a blue color (α), FIG. 2 (b), on the histochemical analysis of the activity of the β-glucoronidase in the transformed protoplasts. FIG. 3 shows the Trametes elegans protoplasts before being submitted to the genetic modification step, FIG. 3(a) the same time the protoplasts that accepted the genetic material and present a blue color (Θ), FIG. 3 (b), in the histochemical analysis of the activity of the β-glucoronidase in the transformed protoplasts. Finally, FIG. 4 shows the Ganoderma lucidum protoplasts before being subjected to the genetic modification step, FIG. 4(a) and at the same time the protoplasts that accepted the genetic material present with a blue color (λ), FIG. 4(b), on the histochemical analysis of the activity of the β-glucoronidase in the transformed protoplasts. For the most part, FIGS. 2, 3 and 4 corroborate that the present invention is successful in the genetic transformation step of 3 different species of basidiomycetes, with high efficiency (Table 2).

### Recovery, Selection, and Proliferation of the Transformed Cell

The objective of the recovery step, selection, and proliferation of the transformed cells (3) in FIG. 1 is to select the cells that received and integrated the genetic material in their genomes (genetically transformed) and proliferate them in liquid or semi-solid culture medium, generating a pure culture line of the species of basidiomycete in question.

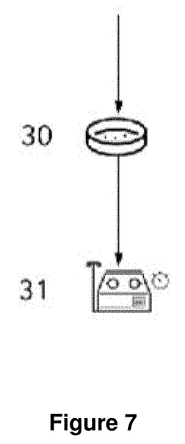

The steps to follow at this point are schematically illustrated in FIG. 7 and are:
1. Directly planting (30) the transformed protoplasts contained in the resulting tubes from the step previous (25), in petri dishes in triplicate, in a semi-solid selective culture medium with the composition shown in table 7:

TABLE 7
Composition of the culture in selective semi-solid

| Component | Composition | |
|---|---|---|
| Carbon source | 5-35% | (w/v) |
| Saccharide | 0.1-15% | (w/v) |
| Getting agent | 0.5-10% | (w/v) |
| Selective antibiotic | 5-500 | mg/L |

The selective semi-solid culture medium composition is presented in Table 7 and should incorporate a carbon source, which includes a high polysaccharides content, that is selected from the group that includes potato and malt extracts, and also a monosaccharide, which is of quick metabolism, that is selected from the group that includes dextrose, maltose and dextrin, a gelling agent selected from the group that includes fitagel, alignato, and agar, and a selective antibiotic, which is capable of destroying the fungi cell, that is selected from the group that includes the Hygromycin B. The selective antibiotic should be preferably Hygromycin B, in view of large inhibition spectrum of the fungi cells. As a consequence, the gene that shows resistance to the same should be included as part of the genetic material to be introduced. Thus, it can be proven in an effective manner the cells that were introduced in the genetic material and that were successfully transformed, proliferating the pure culture.

II. Submitting the plates to a second incubation for the protoplasts genetic transformation (31) to 15-30° C. in the dark for the regeneration of transformed mycelium.

To end the process completely, the objective of this invention is illustrated in FIG. 1 and obtains a pure culture of the genetically transformed strain of the basidiomycete in question.

### Use of the Obtained Strains in Applications of Industrial Interest

The obtained transformed strains can be sub-cultured in a semi-solid, solid, or liquid medium for its posterior application into industrial procedures. For example, in the production of edible mushrooms, fabrication of biomaterials for packaging and delivering, making biomaterials for textiles and footwear, making of biomaterials for the industry of transformation of prime materials, manufacturing of biomaterials for the wood industry, bioleaching based on basidiomycetes, and the production of asset ingredients of interest to industrial areas, as well as other areas.

A person with clear knowledge of the technical field could see that the present invention is of use to a wide range of vectors, like genetic material, the modification of specific characteristics desirable in various species of basidiomycetes fungi, for example, the inclusion commercial and/or industrial interest genes to basidiomycetes used in these sectors. Between the interest features that may be added to said basidiomycetes, are for example: shortening the growth length time, adding specific organoleptic characteristics (color, odor, texture, etc.), modifying the physicochemical properties (hydrophobicity, resistance, etc.), among others modifications.

The selection of vectors is determined just by the desired characteristics in the product obtained by the application of the process, material present, and employment in various vectors does not change the process more than the claims that follow.

Once described and the process explained, the invention can be considered new by comparing it to other property as well as the following claims.

1. A process for generating genetically modified basidiomycetes cells by an inclusion of genetic material to basidiomycetes protoplasts, the method including the steps of:
(a) obtaining mycelia derived protoplasts and placing into petri dishes with a semi-solid culture medium by the introduction of a glucan digestion enzyme, a chitin digestion enzyme, and an osmotic agent in the solution,
(b) genetically transforming the protoplasts by incubating with the genetic material using polyethylene glycol as an adjuvant to inserting the genetic material in the protoplasts, and
(c) selectively recuperating, regenerating, and growing the transformed protoplast and expressing the genetic modification of interest.

2. The process for the genetic modification of basidiomycetes according to claim 1, wherein the step of obtaining the basidiomycetes protoplasts by the use of digestion enzymes over mycelium includes the steps of:
(a) proliferating the mycelium of the required species in petri dishes with semi-solid medium using de-mineralized water as a solvent and sterilizing the solution in an autoclave,
(b) growing the strains at 15-30° C. in the dark for 7 days or until the mycelium completely covers the petri dish,
(c) placing the mycelium equivalent to ⅛ of the petri dish in a Eppendorf tube of 2 mL,
(d) adding to the tube a volume equal to a weight of the mycelium, an osmotic agent solution 10-30% (w/v) to regulate the osmotic pressure,
(e) submitting the tube to a first incubation for the collection of protoplasts to 20-31° C. for 20-40 minutes,
f) adding to the tube 1.5 volume of a digestion solution and set the pH 5.5-6.3, (g) submitting the tube to a second incubation for the collection of protoplasts to 25-37° C. with an agitation of 120-170 rpm by a period of 20-45 minutes,
(h) centrifuge the tube at 1000-1800 rpm for 1-5 minutes,
i) discarding 70-90% of a supernatant, and
j) re-suspending the remaining volume in 50-100 micro liters of osmotic agent solution 10-30% (w/v).

3. The process for genetic modification of basidiomycetes according to claim 1, wherein the semi-solid medium has the following composition:

| component | Concentration (w/v) |
|---|---|
| carbon source | 5-35% |
| saccharide | 0.1-15% |
| gelling agent | 0.5-10%. |

4. The process for genetic modification of basidiomycetes according to claim 3, wherein the carbon source of the semi-solid medium has a high content of polysaccharides and includes amylase, amylopectin, or maltodextrin, derived from potato or malt extracts.

5. The process for genetic modification of basidiomycetes according to claim 3, wherein the saccharide of the semi-solid medium is selected from the group including dextrose, maltose, or dextrin.

6. The process for genetic modification of basidiomycetes according to claim 3, wherein the gelling agent is agar.

7. (canceled)

8. The process for genetic modification of basidiomycetes according to claim 1, wherein the osmotic agent contains between 11 and 15% (w/v) of an osmotic agent.

9. (canceled)

10. The process for genetic modification of basidiomycetes according to claim 1, wherein the osmotic agent is selected from the group that includes sorbitol or citric acid.

11. (canceled)

12. The process for genetic modification of basidiomycetes according to claim 1, wherein the digestion solution has a of pH 5.7-6.1 and the following composition:

| component | Concentration | |
|---|---|---|
| glucan digestion enzyme | 1-10% | (w/v) |
| chitin digestion enzyme | 1-5% | (w/v) |
| osmotic agent | 0.1-1 | m |
| Shock absorbing agent | 10-100 | mM |
| cytoplasmic membrane stabilizing agent | 1-10 | mM |

13. The process for genetic modification of basidiomycetes according to claim 12, wherein the glucan digestion enzyme includes cellulose, glucana, or cellobiohydrolase.

14. The process for genetic modification of basidiomycetes according to claim 12, wherein the chitin digestion enzyme is selected from the group that includes pectinase, polygalacturonase, or chitinase.

15. The process for genetic modification of basidiomycetes according to claim 12, wherein the osmotic agent in the digestion solution for the collection of protoplasts of basidiomycetes is selected from the group that includes the sorbitol or the citric acid.

16. The process for genetic modification of basidiomycetes according to claim 12, wherein the shock absorbing agent is selected from the group that includes HEPES, the Tris, and TAE.

17. The process for genetic modification of basidiomycetes according to claim 12, wherein the stabilizing cytoplasmic membrane agent is selected from the group that includes calcium chloride.

18. (canceled)

19. (canceled)

20. (canceled)

21. (canceled)

22. The process for genetic modification of basidiomycetes according to claim 1, wherein the genetically transformation step to the protoplasts, includes the steps of:
(a) adding to the tube with the obtained protoplasts on the protoplasts obtaining step, 1-100 micro grams of the generic material,
(b) submitting the tubes to the first incubation to genetically transform the protoplast by 1-10 minute to temperature environment (20-25° C.),
(c) adding to the tube 2-10 micro liter solution of adjuvant,
(d) submitting the tube to a second incubation to genetically transform to the protoplasts to 25-37° C. during 5-15 minutes,
(e) adding 1-2 mL of the osmotic agent solution, and
f) submitting the tube to a third incubation to genetically transform to the protoplasts during 40-60 hour in the dark at 15-30° C.

23. (canceled)

24. (canceled)

25. The process for genetic modification of basidiomycetes according to claim 1, wherein the adjuvant solution in the step where the protoplasts are genetically transformed and has the following composition:

| component | concentration (w/v) |
|---|---|
| polyethylene glycol | 20-50% |
| osmotic agent | 10-30% |

26. (canceled)

27. (canceled)

28. (canceled)

29. (canceled)

30. (canceled)

31. The process for genetic modification of basidiomycetes according to claim 22, wherein the third incubation to genetically transform to the protoplasts in dark is carried out at 23° C. for 48 hours.

32. The process for genetic modification of basidiomycetes according to claim 1, wherein the selectively recuperating, regenerating, and growing steps of the cell transformed includes the steps of:
a) directly planting the obtaining transformed protoplasts in the tubes of the genetically transforming the protoplast to the petri dish in triplicate, in the selective semi-solid medium, and
(b) submitting the dishes to an incubation for recovery, regeneration, and proliferation of cells transformed to 15-30° C. in dark for the regeneration of the transformed mycelium.

33. (canceled)

34. The process for genetic modification of basidiomycetes according to claim 32, wherein the semi-solid culture medium to the selectively recuperating, regenerating, and growing step of the transformed cells has the following composition:

| component | composition | |
|---|---|---|
| carbon source | 5-35% | (w/v) |
| saccharide | 0.1-15% | (w/v) |
| gelling agent | 0.5-10% | (w/v) |
| selective antibiotic | 5-500 | mg/L |

35. (canceled)

36. (canceled)

37. (canceled)

38. The process for genetic modification of basidiomycetes according to claim 34, wherein selective antibiotic is selected from the group that includes hygromycin B, Antimycin A, or Fungicidin.

39. (canceled)

40. (canceled)

41. A product including the basidiomycetes according to claim 1, wherein the product is selected from the group consisting of a fungi food, a manufacturing biomaterial for packaging, biomaterials for textiles and shoes, biomaterials for the industry of transformation matter prima, biomaterials to wood industry, and bioleached based on basidiomycetes, and active ingredients for the industry.
